(19) United States
(12) Patent Application Publication
(10) Pub. No.: US 2019/0151390 A1
(43) Pub. Date: May 23, 2019

(54) PERILLA EXTRACT COMPOSITION

(71) Applicant: INQPHARM GROUP SDN BHD, KUALA LUMPUR (MY)

(72) Inventors: Audrey Li Chin Chew, Kuala Lumpur (MY); Qingwen CHEN, Kuala Lumpur (MY); Bee Kwan TAN, Kuala Lumpur (MY); Volker SCHEHLMANN, Kuala Lumpur (MY); Albrecht Jörg Matthias MILLER, Kuala Lumpur (MY)

(73) Assignee: INQPHARM GROUP SDN BHD, KUALA LUMPUR (MY)

(21) Appl. No.: 16/313,237

(22) PCT Filed: Jun. 22, 2017
(86) PCT No.: PCT/MY2017/000024

§ 371 Date: Dec. 26, 2018

(30) Foreign Application Priority Data
Jun. 28, 2016 (MY) PI 2016702386

(51) Int. Cl. A61K 36/535; A61K 36/53; A61K 36/185; A61K 36/481; A61K 36/71; A61K 36/48; A61K 9/48; A61K 9/06; A61K 9/20; A61K 47/10; A61K 47/24; A61K 47/34; A61K 47/32; A61K 47/12; A61K 31/55; A61K 47/46

(52) U.S. Cl. CPC A61K 36/535; A61K 47/46; A61K 36/185; A61K 36/481; A61K 36/71; A61K 36/48; A61K 9/4858; A61K 9/06; A61K 9/2009; A61K 9/2013; A61K 9/2054; A61K 47/10; A61K 47/24; A61K 47/34; A61K 47/32; A61K 47/12; A61K 31/55; A61K 36/53

(57) ABSTRACT

Compositions and methods for use in inhibiting mast cell activation and/or treatment or prevention of disorders associated with mast cell activation or degranulation. The compositions and methods of use include a Perilla spp. extract, such as in combination with another plant extract. The second plant extract desirably adds anti-inflammatory and/or anti-allergy properties to the compositions.

### BACKGROUND OF THE INVENTION

### Field of the Invention

The present invention relates generally to the use of a combination of a Perilla spp. extract and another plant extract (e.g. derived from Sesamum spp.) to inhibit activation of mast cells. In particular, the present invention relates to the use of a combination of a Perilla spp. extract and another plant extract (e.g. derived from Sesamum spp.) to prevent and/or treat a disorder associated with mast cell activation or degranulation such as an allergic disorder, anaphylaxis, an autoimmune disorder, or symptoms associated with mast cell activation disorder, mast cell clonal disorder or mast cell neoplastic disorder.

### Discussion of Related Art

Mast cells are granulocytes derived from myeloid stem cells. Mast cells are activated by allergens through cross-linking with IgE receptors on the surface of the cells, physical injury through damage-associated molecular pattern molecules, pathogen-related agents through pathogen-associated molecular pattern molecules, various compounds with their associated G-protein coupled receptors and complement proteins. Activation of mast cells causes a complex intracellular signalling pathway to occur, which results in degranulation of mast cells. This is where molecules stored in the granules of the mast cells are released outside of the cell. This includes, for example, serine proteases (e.g. tryptase), histamine, serotonin and proteoglycans (e.g. heparin). Mast cells play a role in the immune and neuroimmune systems, wound healing, angiogenesis and blood-brain barrier function. Mast cells have thus been implicated in numerous diseases.

Therefore, there is an ongoing need for compositions that inhibit mast cells, which may, for example, have prophylactic and/or therapeutic effects on disorders associated with mast cell activation and/or degranulation.

### SUMMARY OF THE INVENTION

The present invention aims, at least in part, to provide a composition having a specific combination of active ingredients, which acts to inhibit mast cell activation. In certain embodiments, the present invention aims to provide a composition having a specific combination of active ingredients, which act synergistically, to inhibit mast cell activation and/or mast cell degranulation and/or release of histamine from mast cells.

According to a first aspect, there is provided a composition comprising a Perilla spp. extract and another plant extract.

The other plant extract may, for example, be an anti-inflammatory and/or an anti-allergic plant extract. The other plant extract may, for example be derived from Nigella spp., Urtica spp., Astragalus spp., Petasites spp., Citrus spp., Uncaria spp., Lavandula spp., Mentha spp., Eucalyptus spp., Matricaria spp., Rosmarinus spp., Curcuma spp., Allium spp. or Sesamum spp. In certain embodiments, the other plant extract is derived from Sesamum spp.

The Perilla spp. extract and/or the other plant extract may be an oil. In certain embodiments, the composition comprises perilla oil and sesame oil.

The Perilla spp. extract and other plant extract may be present in the composition in a weight ratio ranging from about 1:10 to about 10:1.

The composition may, for example, be a solid preparation such as a tablet, capsule, caplet, draged, lozenge, granule, powder, pellet, bead, cachet or bolus. The composition may, for example, be a liquid preparation such as an elixir, syrup, suspension, spray, emulsion, lotion, solution or tincture. The composition may, for example, be a semi solid preparation such as ointment, cream, paste, gel or jelly.

According to a second aspect, there is provided a pharmaceutical composition comprising a composition according to the first aspect and a pharmaceutically-acceptable carrier, excipient, diluent and/or additive.

The pharmaceutically acceptable carrier, excipient and/or diluent for use in the pharmaceutical composition can be selected from the group consisting of fillers, binders, surfactants, preservatives, colourants, flavouring substances, pH regulators, regulators of the osmotic activity and salt-forming groups.

The composition may, for example, inhibit activation of mast cells. The composition may, for example, inhibit degranulation of mast cells. The composition may, for example, inhibit release of histamine from mast cells.

Thus, according to a third aspect, there is provided the use of a composition comprising a Perilla spp. extract and another plant extract to inhibit activation of mast cells.

According to a fourth aspect, there is provided a therapeutic or non-therapeutic or in vitro method of inhibiting activation of mast cells, the method comprising administering a composition comprising a Perilla spp. extract and another plant extract to a subject.

According to a fifth aspect, there is provided a composition comprising a Perilla spp. extract and another plant extract for use in a therapeutic method, for example for use in a therapeutic method of inhibiting mast cell activation.

According to a sixth aspect, there is provided the use of a Perilla spp. extract and another plant extract in the manufacture of a medicament or pharmaceutical composition.

The composition may, for example, inhibit degranulation of mast cells. The composition may, for example, inhibit release of histamine from mast cells.

The composition may, for example, reduce histamine release by at least about 20% or by at least about 40% or by at least about 50%. For example, the composition may reduce the concentration of histamine in the tissue of a subject by at least about 20% or by at least about 40% or by at least about 50%.

The composition may, for example, prevent and/or treat a disorder associated with mast cell activation and/or mast cell degranulation. The composition may, for example, prevent and/or treat symptoms associated with a mast cell activation disorder (e.g. mast cell activation syndrome (MCAS)), an allergic disorder (e.g. allergic asthma, allergic rhinitis, conjunctivitis, angioedema, urticaria, eczema, food allergy, drug allergy and insect allergy), anaphylaxis, an autoimmune disorder, symptoms associated with a mast cell clonal disorder (e.g. mastocytosis) or symptoms associated with a mast cell neoplastic disorder (e.g. mastocytoma, mast cell sarcoma and mast cell leukemia).

According to a seventh aspect, there is provided a method for making a composition comprising a Perilla spp. extract and another plant extract, the method comprising combining the Perilla spp. extract and the other plant extract.

Embodiments of the invention will be further described in the detailed description. Any embodiment described herein or any combination of embodiments described herein is applicable to any one or more aspects of the present invention unless clearly contradicted by context.

### BRIEF DESCRIPTION OF SEVERAL VIEWS OF THE DRAWINGS

FIG. 1 is a graph showing the percentages of histamine reduction from HMC-1 cell line when treated with perilla seed oil, sesame seed oil, cromolyn and the combination, as compared to the respective controls.

FIG. 2 is a graph showing the percentages of histamine reduction from HMC-1 cell line when treated with the combination and Cromolyn, compared to the averaged histamine values of perilla and sesame oils.

### DETAILED DESCRIPTION OF THE INVENTION

The present invention is based on, at least in part, the surprising finding that a combination of a Perilla spp. extract and another plant extract can inhibit mast cell activation. For example, embodiments of the present invention are based on the surprising finding that a Perilla spp. extract and another plant extract may work synergistically to inhibit mast cell activation. In certain embodiments, the combination of a Perilla spp. extract and another plant extract can be used to treat disorders associated with mast cell activation and/or mast cell degranulation.

Hereinafter, the invention shall be described according to preferred embodiments of the present invention and by referring to the accompanying description and drawings. However, it is to be understood that limiting the description to the preferred embodiments of the invention is merely to facilitate discussion of the present invention and it is envisioned that those skilled in the art may devise various modifications without departing from the scope of the appended claim.

The terms generally used hereinbefore and hereinafter have for preference the meanings indicated below, unless indicated otherwise, whereby more specific meanings may be used independently of one another in preferred embodiments of the present inventions instead of the general definitions, these more specific significances describing especially preferred embodiments of the invention.

Where the term “at least one” or “one or more” occurs hereinbefore and hereinafter, this signifies in particular one to ten, for preference one to three, and in particular one or, further, two of the features enumerated, such as components. Where ranges are indicated, such as weight percentage ranges, these include the limit values indicated; thus, for example, “between X and Y” signifies “from and including X up to and including Y”.

The term “product” or “composition” may mean in particular a pharmaceutical product or pharmaceutical composition in the sense of a formulation, whereby this term is not restricted to pharmaceutical products suitable for registration, or a medical product or fictitious pharmaceuticals. The “product” or “composition” may refer to a nutraceutical product in the sense of a formulation.

The term “anti-allergic” is to be understood to relate to an agent or measure that prevents, inhibits, or alleviates an allergic reaction.

The term “anti-inflammatory” is to be understood to relate to an agent or measure that prevents, inhibits, or alleviates an inflammatory reaction.

The term “therapeutic treatment” or “therapeutic method”, also includes prophylaxis and the alleviation of symptoms in a subject, although not cosmetic treatments. This relates to therapeutic treatment, and in particular prophylaxis or alleviation, of disorders relating to mast cells, for example disorders relating to mast cell activation and/or mast cell degranulation.

The components referred to hereinbefore and hereinafter are in particular selected from among those such as are listed in pharmacopoeia, e.g. in the US Pharmacopoeia National Formulary, the Pharmacopoea Europea, the Pharmacopoea Helvetica, the British Pharmacopoeia, the German Pharmacopoeia, the Japanese Pharmacopoeia, the Chinese Pharmacopoeia, or supplements, such as by way of decrees.

The present invention relates to a composition comprising a Perilla spp. extract and another plant extract. The composition may, for example, comprise one or more further plant extracts. For example, the composition may comprise a third or fourth or fifth or sixth plant extract. The composition may, for example, consist essentially of or consist of a Perilla spp. extract and another plant extract. The composition may, for example, consist essentially of or consist of a Perilla spp. extract and another plant extract and a third plant extract and optionally a fourth, fifth or sixth plant extract.

The term “extract” encompasses aqueous extract, non-aqueous extract, alcoholic extracts, evaporation, distillation (e.g. by water, steam, hydro diffusion, cohobation, rectification, fractional, etc), expression/cold pressing, supercritical CO₂ extraction, dilutions, plant concentrates, plant oils, plant macerations, plant exudates, plant resins, plant powders and plant granules and any combination of two or more thereof. However, the invention should not be construed as being limited to such embodiments.

The Perilla spp. extract may, for example, be derived from Perilla frutescens (also known as perilla, Chinese basil and beefsteak), Perilla citrodora, Perilla hirtella, Perilla setoyensis or a combination of two or more thereof.

The other plant extract and the optional third, fourth, fifth or sixth plant extracts may, for example, each independently be an anti-inflammatory or an anti-allergic plant extract. The other plant extract and the optional third, fourth, fifth or sixth plant extracts may, for example, each independently be a mast cell stabilizing extract. The other plant extract and the optional third, fourth, fifth or sixth plant extracts may, for example, each independently be a medicinal or therapeutic or nutritional or health benefiting plant extract.

The other plant extract and the optional third, fourth, fifth or sixth plant extracts may, for example, each be independently derived from Nigella spp. (e.g. Nigella sativa which is also known as black cumin, black caraway or fennel seed), Urtica spp. (e.g. nettle), Astragalus spp. (e.g. milk vetch), Petasites spp. (e.g. butterbur), Citrus spp. (e.g. lemon), Uncaria spp. (e.g. Cat's claw), Lavandula spp. (e.g. lavender), Mentha spp. (e.g. peppermint), Eucalyptus spp. (e.g. Eucalyptus globulus which is also known as Tasmanian blue gum, southern blue gum or blue gum), Matricaria spp. (e.g. German chamomile), Rosmarinus spp. (e.g. rosemary), Curcuma spp. (e.g. turmeric), Allium spp. (e.g. garlic), Sesamum spp. or a combination of any two or more thereof. In certain embodiments, the other (second) plant extract is derived from Sesamum spp.

The Perilla spp. extract, the other plant extract and the optional third, fourth, fifth and sixth plant extracts may, for example, each be independently obtained from the seed root, leaf, or whole of the plant as listed in the preceding description. In certain embodiments, the plant extracts are each independently (e.g. all) derived from the seed of the plant.

In certain embodiments, the Perilla spp. extract is perilla oil and the other plant extract is sesame oil. In certain embodiments, the Perilla spp. extract is perilla oil derived from the seed of the plant of Perilla spp., and the other plant extract is sesame oil. In certain embodiments, the Perilla spp. extract is Perilla frutescens seed oil and the other plant extract is Sesamum indicum seed oil.

In certain embodiments, the Perilla spp. extract is perilla oil and the other plant extract is nigella oil, eucalyptus oil, peppermint oil, lavender oil, lemon oil, chamomile oil, frankincense oil, basil oil, tea tree oil, clove oil or a combination of any two or more thereof.

The Perilla spp. extract and the other plant extract may, for example, be present in the composition in a weight ratio ranging from about 1:10 to about 10:1. For example, the Perilla spp. extract and the other plant extract may be present in the composition in a weight ratio from about 1:9 to about 9:1 or from about 1:8 to about 8:1 or from about 1:7 to about 7:1 or from about 1:6 to about 6:1, or from about 1:5 to about 5:1, or from about 1:4 to about 4:1, or from about 1:3 to about 3:1, or from about 1:2 to about 2:1. For example, the Perilla spp. extract and the other plant extract may be present in a weight ratio of about 1:1. For example, the Perilla spp. extract and the other plant extract may be present in a weight ratio ranging from about 1:1 to about 10:1 or from about 1:1 to about 8:1 or from about 1:1 to about 5:1 or from about 1:1 to about 2:1.

In certain embodiments, the Perilla spp. extract is perilla oil and the other plant extract is sesame oil and the weight ratio of perilla oil to sesame oil is in the range of about 1:10 to 10:1, for example, from about 1:9 to about 9:1, or from about; 1:8 to about 8:1, or from about 1:7 to about 7:1, or from about 1:6 to about 6:1, or from about 1:5 to about 5:1, or from about 1:4 to about 4:1, or from about 1:3 to about 3:1, or from about 1:2 to about 2:1, or about 1:1. For example, the weight ratio of perilla oil to sesame oil may range from about 1:1 to about 10:1 or from about 1:1 to about 8:1 or from about 1:1 to about 5:1 or from about 1:1 to about 2:1.

The Perilla spp. extract and the other plant extract may, for example, be present in the composition in an amount by weight from about 5% to about 99.5% or from about 10% to about 95% or from about 20% to about 90% or from about 30% to about 80% or from about 40% to about 70% or from about 50% to about 60%. For example, the Perilla spp. extract and the other plant extract may, for example, be present in the composition in an amount by weight from about 20% to about 60% or from about 25% to about 55% or from about 30% to about 50% or from about 35% to about 45%.

The composition of the present invention may be administered in the form of a composition comprising any suitable additional component. The composition may, for example, be a pharmaceutical composition (medicament), suitable for oral, nasal, topical, suppository, intravenous, intradermal, transdermal, transmucosal or inhalation administration. The composition may alternatively be a nutraceutical composition, for example, a foodstuff, food supplement, dietary supplement, health supplement, meal replacement product, beverage, beverage supplement, food additive, animal feed or feed additive.

The compositions may take the form, for example, of solid preparations including tablets, capsules, caplets, drageés, lozenges, granules, powders, pellets beads, cachets and boluses; liquid preparations including elixirs, syrups, suspensions, sprays, emulsions, lotions, solutions and tinctures; or semi solid preparations including ointments, creams, pastes, gels and jellies. Techniques and formulations generally may be found in Remington, The Science and Practice of Pharmacy, Mack Publishing Co., Easton, Pa., latest edition.

The term “pharmaceutical composition” or “medicament” in the context of this invention means a composition comprising (a pharmaceutically effective amount of) a Perilla spp. extract and another plant extract, and additionally one or more pharmaceutically acceptable carriers and/or excipients. The pharmaceutical composition may further contain ingredients selected from, for example, diluents, adjuvants, excipients, vehicles, counterions, preserving agents, fillers, binders, disintegrating agents, wetting agents, emulsifying agents, suspending agents, sweetening agents, flavouring agents, colouring agents, preserving agents, gelling agents, perfuming agents, antibacterial agents, antifungal agents, lubricating agents, coating agents, encapsulating agents, buffering agents, surfactants and dispersing agents, depending on the nature of the mode of administration and dosage forms.

In solid dosage forms of the invention (e.g. for oral or topical administration), the active ingredient(s) may be mixed with one or more pharmaceutically acceptable carriers, such as dicalcium phosphate, and/or any of the following: diluents, fillers or extenders, such as starches, lactose, sucrose, glucose, mannitol, microcrystalline cellulose, maltodextrine and/or silicic acid; binders, such as, for example, hydroxypropylcellulose, hypromellose, hydroxypropyl methylcellulose, carboxymethylcellulose, gelatine, polyvinyl pyrrolidones, polyvinyl acetate, sucrose and/or acacia; disintegrating agents, such as starch, for example, potato or tapioca starch, starch derivatives such as sodium starch glycolate, crospolyvinylpyrollidone, calcium carbonate, croscarmellose sodium, alginic acid, and certain silicates; lubricants, such as talc, calcium stearate, magnesium stearate, stearic acid, sodium sulfate stearyl fumarate, solid polyethylene glycols, solubiliser such as sodium lauryl sulfate, glidants such as silicon dioxide, flavouring and colouring agents and mixtures thereof.

Tablets, and other solid dosage forms of the compositions of the invention, may optionally be prepared with coatings and shells, such as enteric coatings and other coatings well known in the pharmaceutical-formulation art. They may also be formulated so as to provide slow or controlled release of the active ingredient(s) therein using, for example, natural and synthetic polymers such as hydroxypropylmethyl cellulose methacrylates, methacrylic acid copolymers (e.g. methyl acrylate-methacrylic acid copolymers and methyl methacrylate-methacrylic acid copolymers), shellac, ethylcellulose, cellulose acetate phthalate, cellulose acetate trimellitate, polyvinyl acetate phthalate, cellulose acetate succinate, hydroxyl propyl methyl cellulose acetate succinate, sodium alginate, waxes, fatty acids, zein, respectively, in varying proportions to provide the desired release profile, other polymer matrices, liposomes and/or microspheres may also be used. These compositions may also optionally contain colourants and/or opacifying agents and may be of a composition such that they release the active ingredient(s) only, or preferentially, in a certain portion of the gastrointestinal tract, optionally, in a delayed manner.

The compositions may comprise no more than about 50% w/w of pharmaceutically acceptable carrier and/or excipient, for example, no more than about 45% w/w of pharmaceutically acceptable carrier and/or excipients, or no more than about 40% of w/w pharmaceutically acceptable carrier and/or excipients, or no more than about 35% w/w of pharmaceutically acceptable carrier and/or excipients. For example, the pharmaceutical composition may comprise at least about 1% w/w, or at least about 10% w/w, or at least about 15% w/w, or at least about 20% w/w, or at least about 25% w/w, or at least about 30% w/w of pharmaceutically acceptable carrier and/or excipients.

Liquid or semi-solid form preparations include solutions, suspensions, and emulsions, for example, water or water-propylene glycol solutions (e.g. for oral or topical administration). Liquid or semi-solid preparations can also be formulated in solution in aqueous polyethylene glycol solution or an ointment base as known in the art (e.g. petrolatum). In certain embodiments, the active ingredient(s), i.e., plant extracts, may be mixed with one or more pharmaceutically acceptable carriers, such as water and/or any of the following: solvent such as propylene glycol, isopropyl myristate, alcohol; humectant such as glycerol; sweeteners such as liquid glucose, corn syrup and sucrose; artificial sweeteners such as aspartame, stevia and sucralose; preservatives such as phenoxyethanol, caprylyl glycol, ethylhexylglycerin, benzalkonium chloride, potassium sorbate, disodium EDTA, benzoates and parabens; viscosity modifiers/thickeners such as gums, soybean oil, polyacrylates (e.g. sodium polyacrylate, Carbopol 980 polymer) and alginates; buffering agents such as monopotassium phosphate, di-potassium hydrogen phosphate and glycerin; gelling or emulsifying agents such as bentonite, xanthan gum, carrageenan, polysorbate 80, polysorbate 20, gelatin, cetearyl alcohol, lecithin, glycerol monostearate, flavouring agents in aqueous or in oil forms such as vanilla oil; cooling agents such as menthol; soothing or moisturising agents such as Aloe vera, hyaluronic acid; antioxidants such as tocopherols and citric acid; colouring agents; pH regulators and surfactants.

Also included are solid form preparations, for example, tablets, capsules, granules and powder, which are intended to be converted, shortly before use, to liquid form preparations for oral administration. Such liquid forms include solutions, suspensions, and emulsions. These particular solid form preparations are most conveniently provided in unit dose form and as such are used to provide a single liquid dosage unit. Alternatively, sufficient solids may be provided so that multiple individual liquid doses may be reconstituted when required, by measuring predetermined volumes of the solid form preparation as with a spoon, or other measuring device. The solid form preparations intended to be converted to liquid form may contain, in addition to the active material, flavourings, colourants, stabilizers, buffers, artificial and natural sweeteners, dispersants, thickeners, solubilising agents, and the like. The liquid utilized for preparing the liquid form preparation may be water, isotonic water, juices, milk, ethanol, and the like as well as mixtures thereof.

The terms “food”, “foodstuff”, “food supplement”, “dietary supplement”, “health supplement”, “meal replacement product”, “beverage” and “beverage supplement” used herein have the normal meanings for those terms, and are not restricted to pharmaceutical preparations. Other composition forms are also included within the present invention. These may, for example, include, a foodstuff precursor such as a rehydratable powder or a beverage precursor such as a powder dispersible in water, milk or other liquid.

Also included are solid form preparations which are intended to be combined with a food or foodstuff before oral consumption. The solid form preparations may be mixed into the food or foodstuff or applied to the food or foodstuff, e.g., by sprinkling onto the food or foodstuff. Such solid forms include powders, granules, pellets and the like.

In certain embodiments, the food or foodstuff, and the like, comprises from about 0.1 wt. % to about 50 wt. % of the composition of the invention described herein, based on the total weight of the food or foodstuff, for example, from about 0.1 wt. % to about 40 wt. %, or from about 0.1 wt. % to about 30 wt. %, or from about 0.1 wt. % to about 20 wt. %, or from about 0.1 wt. % to about 15 wt. %, or from about 0.1 wt. % to about 10 wt. %, or from about 0.1 wt. % to about 8 wt. %, or from about 0.1 wt. % to about 6 wt. %, or from about 0.1 wt. % to about 4 wt. %, or from about 0.1 wt. % to about 2 wt. % of the composition of the invention described herein. In certain embodiments, the food or foodstuff, and the like, comprise at least about 0.2 wt. % of the compositions of the invention described herein, based on the total weight of the food or foodstuff, for example, at least about 0.5 wt. %, or at least about 1 wt. %, or at least about 5 wt. % of the composition of the invention described herein.

The composition may further comprise other biologically active agents, for example, biologically active agents suitable for inhibiting mast cell activation or suitable for preventing or treating a disease or disorder associated with mast cell activation or mast cell degranulation. For example, the composition may further comprise other anti-allergy active agents.

The composition or pharmaceutical composition may further comprise a nutrient ingredient selected from the group consisting of vitamins and minerals, and combinations thereof. The vitamin may be any one or more of vitamin A, vitamin D, vitamin E, vitamin K, thiamine, riboflavin, pyridoxine, cyanocobalamin, carotenoids (including beta-carotene, zeaxanthin, lutein and lycopene), niacin, folic acid, pantothenic acid, biotin, vitamin C, choline, inositol, and salts and derivatives thereof. The mineral may be any one or more of calcium, phosphorous, magnesium, iron, zinc, manganese, copper, cobalt, boron, iodine, sodium, potassium, molybdenum, selenium, chromium, fluorine and chloride.

The composition or pharmaceutical composition may further comprise dietary fibre of plant and/or non-plant origin. The term “dietary fibre” used herein has its normal meaning for this term. It is generally regarded as the indigestible portion of food derived from plants. Typically, there are two main components of dietary fibre: soluble fibre, which dissolves in water, and insoluble fibre, which does not dissolve in water. Soluble fibres include inulin, chitosan, gum acacia, guar gum, low-methoxy and high-methoxy pectin, oat and/or barley beta glucans, carrageenan, psyllium, cyclodextrin, and derivatives thereof. Insoluble fibres include oat hull fibre, pea hull fibre, soy hull fibre, soy cotyledon fibre, sugar beet fibre, cellulose, corn bran and derivatives thereof.

The compositions and pharmaceutical compositions described herein may be used in various therapeutic, non-therapeutic and in vitro applications. For example, the compositions and pharmaceutical compositions described herein may be used to inhibit mast cell activation and/or mast cell degranulation and/or release of histamine from mast cells. For example, the compositions and pharmaceutical compositions described herein may be used in an in vitro method or in an in vivo method. The methods may comprise administering the composition or pharmaceutical composition described herein to a subject.

According to one aspect of the present invention, there is provided a composition according to any aspect or embodiment described herein for use in a therapeutic or non-therapeutic method for inhibiting mast cell activation (a therapeutic or non-therapeutic method for mast cell stabilization). The method may, for example, comprise contacting the mast cells with a composition according to any aspect or embodiment described herein.

In certain embodiments, the plant extract components have a synergistic effect on inhibiting mast cell activation and/or mast cell degranulation and/or release of histamine from mast cells.

Mast cells are activated by allergens through cross-linking with IgE receptors on the surface of the cells, physical injury through damage-associated molecular pattern molecules, pathogen-related agents through pathogen associated molecular pattern molecules, various compounds with their associated G-protein coupled receptors and complement proteins. Activation of a mast cell causes a complex intracellular signalling pathway to occur, which results in degranulation of the mast cells. This is where molecules stored in the granules of the mast cell are released outside of the cell. This includes, for example, serine proteases (e.g. tryptase), histamine, serotonin and proteoglycans (e.g. heparin). Inhibiting activation of mast cells may, for example, include any one or more of these steps. For example, the compositions disclosed herein may be useful in inhibiting mast cell degranulation. For example, the compositions disclosed herein may be useful in inhibiting release of histamine from the mast cell.

Thus, in certain embodiments, the composition according to any aspect or embodiment may reduce histamine release by at least about 20%. For example, the composition may reduce histamine release by at least about 30% or at least about 40% or at least about 50% or at least about 60% or at least about 70% or at least about 75% or at least about 80% or at least about 85% or at least about 90% or at least about 95%. For example, the composition may reduce histamine release by up to 100%, for example up to 99% or up to 98%. This may, for example, be determined by measuring histamine content in cell culture medium, for example as described in the examples below.

According to further aspects, there is provided a composition according to any aspect or embodiment of the present invention for use in preventing and/or treating a disorder associated with mast cells, for example a disorder associated with mast cell activation and/or mast cell degranulation. For example, there is provided a composition according to any aspect for use in preventing and/or treating an allergic disorder and/or anaphylaxis and/or an autoimmune disease, or symptoms associated with mast cell activation disorder, a mast cell clonal disorder and/or a mast cell neoplastic disorder. In certain embodiments, the disorder is not a nasal disorder. In certain embodiments, the disorder is not an allergic disorder of the nasal cavity. In certain embodiments, the disorder is not seasonal allergic rhinitis or perennial allergic rhinitis.

According to a further aspect, there is provided the use of a Perilla spp. extract and another plant extract in the manufacture of a medicament or pharmaceutical composition. The medicament or pharmaceutical composition may, for example, be for preventing and/or treating an allergic disorder and/or anaphylaxis and/or an autoimmune disease, or symptoms associated with mast cell activation disorder, a mast cell clonal disorder and/or a mast cell neoplastic disorder.

Mast cell activation disorders encompass disorders caused by or associated with inappropriate activation of mast cells. The classification of mast cell activation disorders was laid out in Akin et al. “Mast cell activation syndrome: Proposed diagnostic criteria”, J. Allergy Clin. Immunol. (2010), 126 (6): 1099-1104. For example, the mast cell activation disorder may be mast cell activation syndrome (MCAS).

Allergic disorders encompass disorders caused by hypersensitivity of the immune system. Generally, allergic disorders are caused by hypersensitivity to an allergen that causes little reaction in most subjects. Allergens that may be related to allergic disorders may be derived from, for example, foods, latex, medications, pollen and other animals (e.g. animal hair, insect stings). Allergic disorders encompass allergic disorders of the nasal cavity, including seasonal allergic rhinitis and perennial allergic rhinitis. Allergic disorders also include allergic conjunctivitis, allergic contact dermatitis, atopic dermatitis and allergic asthma. Anaphylaxis is a serious allergic reaction. In certain embodiments, the allergic disorder is not an allergic disorder of the nasal cavity (e.g. is not seasonal allergic rhinitis and/or is not perennial allergic rhinitis).

Autoimmune disorders encompass disorders caused by or associated with an abnormal immune response against a substance that is normally present in the body. Autoimmune disorders include, for example, allergic asthma, Addison disease, celiac disease, dermatomyositis, Graves disease, Hashimoto thyroiditis, multiple sclerosis, myasthenia gravis, pernicious anemia, reactive arthritis, rheumatoid arthritis, Sjogren syndrome, systemic lupus erythematosus, bullous pemphigoid and type I diabetes. For example, autoimmune disorders include autoimmune, inflammatory disorders of the joints and skin.

Mast cell clonal disorders encompass disorders caused by or associated with the presence of an increased number of mast cells in comparison to a normal (healthy) subject. For example, mast cell clonal disorders include mastocytosis.

Mast cell neoplastic disorders encompass disorders caused by or associated with abnormal growth of mast cells. For example, mast cell neoplastic disorders includes mastocytomas, mast cell sarcoma and mast cell leukemia.

The composition according to any aspect or embodiment may reduce histamine concentration in the tissue of a subject by at least about 20%. For example, the composition may reduce histamine concentration in the tissue of a subject by at least about 30% or at least about 40% or at least about 50% or at least about 60% or at least about 70% or at least about 75% or at least about 80% or at least about 85% or at least about 90% or at least about 95%. For example, the composition may reduce histamine concentration in the tissue of a subject by up to 100%, for example up to 99% or up to 98%.

Histamine concentration in the tissue of a subject may be measured by any method known to a person skilled in the art. For example, histamine concentration may be determined by microdialysis sampling followed by quantification with commercially available ELISA kits or by HPLC. For example, histamine concentration at a localised site may be determined using tissue samples such as tissue homogenates. For example, histamine concentration may be determined using tissue from a site where an allergen has come into contact with mast cells or a sampling site (e.g. skin, mucosal lining (respiratory tract, gastrointestinal), blood, organs, bone marrow, etc).

The composition according to the first aspect or a pharmaceutical composition according to the second aspect of the invention can further comprise an anti-allergic drug or pharmaceutically active anti-allergic substance, for example an antihistamine or an additional mast cell stabilizer (e.g. cromoglicate, etc). In certain embodiments, the composition or the pharmaceutical composition can further comprise an antihistamine, including H₁-antihistamine (e.g. azelastine, olopatadine, triprolidine, bilastine, cetirizine, pyrilamine etc), H₂-antihistamine (e.g. cimetidine, famotidine, etc) and H₃-antihistamine (e.g. thioperamide, etc). The antihistamine opposes the activity of histamine receptors in a subject and further enhances the inhibitory effect of mast cell activation of the anti allergic oil and oily component (e.g. perilla oil and sesame oil) in the composition or the pharmaceutical composition. In certain embodiments, azelastine can be used as the antihistamine in the composition of the invention, as it has a triple mode of action, i.e. antihistamine effect, mast cell stabilizing effect and anti-inflammatory effect. For example, the composition or the pharmaceutical composition may comprise from about 0.1 wt. % to about 50 wt. %, or from about 0.01 wt. % to about 10 wt. %, or from about 10 wt. % to about 40 wt. %, or from about 20 wt. % to about 30 wt. % of antihistamine by weight of the composition of the invention described herein.

In certain embodiments, the subject is a human. In other embodiments, the subject is a mammal other than a human, such as non-human primates (e.g. apes, monkeys and lemurs), companion animals such as cats or dogs, working and sporting animals such as dogs, horses and ponies, farm animals such as pigs, sheep, goats, deer, oxen and cattle, and laboratory animals such as rodents (e.g. rabbits, rats, mice, hamsters, gerbils or guinea pigs).

In certain embodiments, the composition is administered daily to the subject (e.g. orally or topically administered daily to the subject). The amount of composition administered may be varied depending upon the requirements of the subject. For both therapeutic and non-therapeutic applications, the amount of composition administered may be varied depending upon the desired results, the requirements of the subject and the severity of the condition being treated. Determination of the proper amount/dosage for a particular situation is within the skill of the art. For example, for therapeutic applications a physician or veterinarian having ordinary skill in the art can readily determine and prescribe the effective amount of the pharmaceutical composition required. The total daily amount/dosage may be divided and administered in portions during the day if desired.

In general, a suitable daily dose of active agents in the composition according to the invention will be that amount which is the lowest dose effective to produce the desired effect, for example, a therapeutic effect, and/or to inhibit mast cell activation and/or to inhibit mast cell degranulation and/or to inhibit release of histamine from mast cells. It is contemplated that a wide range of doses may be used, due to the non-toxic nature of the composition. A person of ordinary skill in the art will understand that a suitable dose or dosage will typically vary from subject to subject, and will be dependent on factors such as the severity of health conditions of the subject at the outset of administration of the composition. For example, the dose of active agents (i.e. plant extracts) in the composition may be up to 15 g per day, for example, up to about 10 g per day, or up to about 5 g per day. In certain embodiments, the doses of active agents in the composition is in the range of 100 mg to about 3 g per day, which may be administered as two or three or more sub-doses administered separately at appropriate intervals throughout the day, optionally in unit dosage forms. In certain embodiments, the dose of active agents in the composition may be from about 10 mg to about 3 g of each extract component per day, for example, from about 500 mg to about 3 g of each component per day, or from about 750 mg to about 2.5 g of each component per day, or from about 1000 mg to about 2000 mg of each component per day. In certain embodiments, the composition may be administered two or three times a day, optionally before, with, or after a meal. In certain embodiments, each dose of active agents is no more than about 5 g, for example, no more than about 3 g, for example, no more than about 2.5 g. Each dose of the plant extracts in the composition may be combined with other conventional agents for inhibiting mast cell activation.

Use for the manufacture of a product for the therapeutic treatment may comprise in particular the appropriate procurement of the product (e.g. as a pharmaceutical product in the broader or narrower sense); in other words, the manufacture of the preparation, its introduction in particular into any device for its administration, its packing, and the corresponding provision of instructions for use in therapeutic treatment, for example by way of a package insert and/or printings on the package.

The compositions and pharmaceutical compositions described herein may be prepared by combining a Perilla spp. extract and a second plant extract and optionally any one or more of the other ingredients described herein, such as one or more further plant extracts, dietary fibre, nutrients, biologically active agents and pharmaceutical excipients and/or carriers and/or diluents. The components are combined in suitable amounts to obtain a composition having the desired quantity of each component. Each component may be combined with one or more other components in any order and combination suitable to obtain the desired product. Such methods are well known in the art, for example, methods known in the food industry (e.g. those used in the preparation of health food bars and the like) and methods known in the pharmaceutical industry. The composition may be prepared in the dry solid form, for example, powder form, and subject to further processing step depending on the types of the formulation for the intended finished products. The methods may further comprise a forming step, wherein the mixture is moulded, pressed, spray dried or otherwise formed into a shape (e.g. bar, ball, pellet, clusters, tablet, beads), preferably with dimensions and/or textures suitable for consumption by a human or other mammalian animal of the types described herein. In one of the embodiments, the composition or pharmaceutical composition may be produced in the form of pills, including gel capsules.

The preparations can be manufactured according to conventional and known methods, which include in particular the mixing of the components, if required step by step and/or under movement and/or heating of the fluid, for example by stirring.

The invention will now be described in detail by way of reference only to the following non-limiting examples.

### EXAMPLES

### Example 1: Mast Cell Test

Mast cell lines, i.e. HMC1 cells (provided by Dr Joseph H. Butterfield; Mayo Clinic, Rochester, Minn., USA), were cultured in Isocove's Modified Dulbecco's Medium (IMDM) containing 10% Fetal Bovine Seum (FBS), 2 mM L-glutamine, 50 μM 2-mercaptoethanol, 100 U/mL penicillin and 100 μg/mL streptomycin. Cells were maintained at 37° C. in 5% CO₂ in a humidified atmosphere.

Percentage of living cells was determined by trypan blue staining. A 1:10 dilution with 50 μl cell suspension and 450 μl trypan blue was prepared and the number of living and dead cells was counted in a hemocytometer. 10⁶ living cells were transferred into safe-lock Eppendorf tubes. Cell suspensions were centrifuged (200 g, 5 min) and supernatant was discarded.

The effect of perilla oil, sesame oil and a combination of perilla oil and sesame oil (1:1 ratio) in the vehicle Kolliphor ELP (polyoxyl-35 hydrogenated castor oil) was determined. The positive control Cromolyn in phosphate buffer saline (PBS), the vehicles PBS and ELP were also tested as controls.

After adding 100 μl IgE (100 ng/ml in PBS or ELP) to the cell pellets, cells were incubated for 12 hours over night, at 37° C. Cells were then incubated for 2 hours, at 37° C. with two different concentrations of compounds, i.e. 250 μg/ml and 500 μg/ml. After centrifugation (200 g, 5 min) supernatant was discarded and cell pellet was again incubated for 1 hour, at 37° C. with 100 μl anti-IgE (1 μg/ml in PBS or ELP). For the final incubation time of 6 hours, at 37° C., 300 μl HMC1 media was added. Cells were again centrifuged (200 g, 5 min) and supernatant was collected and stored as 300 μl aliquots at −20° C. for ELISA measurements. Cell pellets were resuspended in 100 μl HMC1 media for determining cell viability.

Viability of the cells (percentage of living cells) was determined by trypan blue staining (1:10 dilution with 50 μl cell suspension and 450 μl trypan blue was prepared and the number of living and dead cells was counted in a hemocytometer).

The concentration of histamine in the collected supernatant was determined by ELISA, using a kit obtained from IBL International according to the manufacturer's guidelines. Each sample was measured twice. Due to expected measuring limits, vehicle (PBS, ELP) treated samples were diluted 1:2.

Statistical analysis was performed with GraphPad Prism (Version 5.01) software (GraphPad Software Inc., San Diego, Calif., USA) using the unpaired two-tailed Students t test. A P value of less than 0.05 was considered statistically significant.

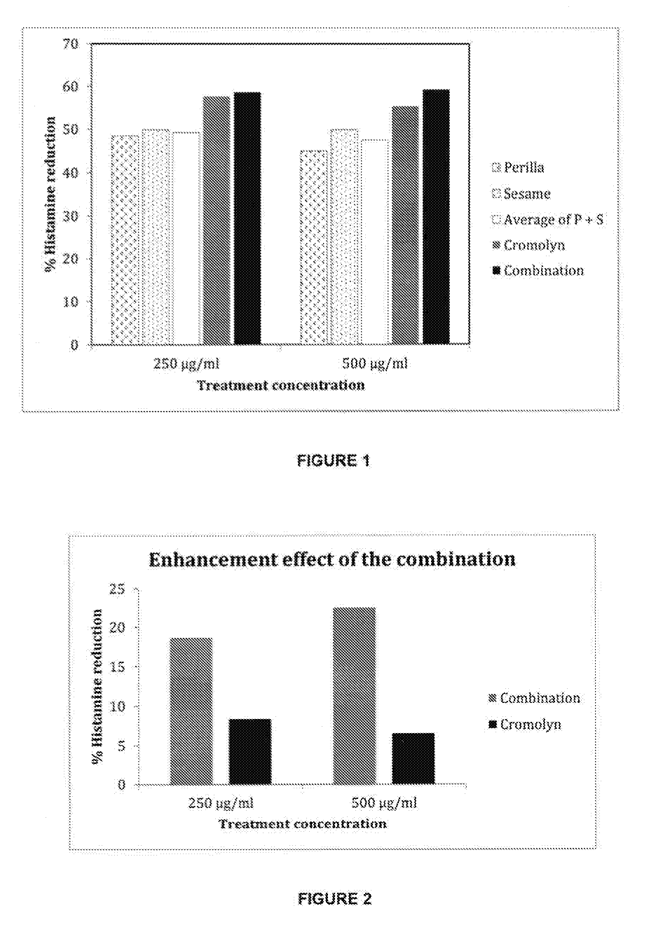

The results are shown in FIGS. 1 and 2, as well as Tables 1 and 2 below.

TABLE 1
Summary of histamine mediator release from HMC-1 cells.
Significant difference compared to negative control was at P < 0.05.

| | | | | | Histamine |
|---|---|---|---|---|---|
| | | | Histamine (ng/ml) | | reduction |
| Treatment | Vehicle | Concentration | mean ± SD (n = 3) | P-value | (%) |
| Perilla oil | Kolliphor | 250 μg/ml | 134.04 ± 7.62 | 0.0009 | 48.46 |
| (P) | ELP | 500 μg/ml | 143.09 ± 6.39 | 0.0012 | 44.98 |
| Sesame oil | | 250 μg/ml | 130.28 ± 6.40 | 0.0008 | 49.91 |
| (S) | | 500 μg/ml | 130.27 ± 11.50 | 0.0010 | 49.91 |
| Average of | | 250 μg/ml | 132.16 | n.a. | 49.18 |
| P + S | | 500 μg/ml | 136.68 | n.a. | 49.18 |
| Combination | | 250 μg/ml | 107.51 ± 12.49 | 0.0046 | 58.66 |
| (1:1 ratio) | | 500 μg/ml | 105.99 ± 0.43 | 0.0031 | 59.25 |
| Control | | n.a. | 260.06 ± 23.62 | n.a. | n.a. |
| Cromolyn | PBS | 250 μg/ml | 121.05 ± 0.73 | 0.0006 | 57.61 |
| | | 500 μg/ml | 127.70 ± 13.32 | 0.0013 | 55.28 |
| Control | | n.a. | 285.54 ± 14.08 | n.a. | n.a. |

TABLE 2
Histamine reduction compared to averaged histamine mediator
release of perilla oil and sesame oil from HMC-1 cells, showing
enhancement effect of the combination. Significant difference
compared to averaged values of perilla oil and sesame
oil was at P < 0.05.

| | | Histamine | |
|---|---|---|---|
| | | reduction | |
| Treatment | Concentration | (%) | P-value |
| Combination | 250 μg/ml | 18.65 | 0.0185 |
| | 500 μg/ml | 22.46 | 0.0092 |
| Cromolyn | 250 μg/ml | 8.41 | 0.0668 |
| | 500 μg/ml | 6.75 | 0.0728 |

Comparing the average histamine release of perilla oil and sesame oil to the combination, the combination showed significantly reduced histamine release and therefore mast cell stabilization at 250 and 500 μg/ml (p=0.0185 and p=0.0092) respectively.

When HMC-1 cells were treated individually with perilla oil or sesame oil, the percentage of histamine reduction achieved was lower than that of Cromolyn. When treated with the combination, the percentage of histamine reduction was higher than treatment with Cromolyn (FIG. 1). Treatment with the combination increased the percentage of histamine reduction by 18.7-22.5% compared to treatment with individual compounds, while treatment with Cromolyn only differed by 6.8-8.4% of histamine reduction compared to treatment with individual compounds.

There is an approximately 2-fold and 3-fold difference between the combination and Cromolyn at concentrations of 250 μg/ml and 500 μg/ml respectively. It can be concluded that the combination had enhanced mast cells stabilizing effect, which increased the percentage of histamine reduction compared to treatment with individual compounds, and reached a final effect that was better than Cromolyn.

### Example 2: Anti-Allergic Formulation

Tables 3-5 show anti-allergic pill formulations in a hard or soft shell capsule.

TABLE 3

| Ingredient | Percentage (%) by weight |
|---|---|
| Perilla oil | 49.75 |
| Sesame oil | 49.75 |
| Tocopherol | 0.5 |

TABLE 4

| Ingredient | Percentage (%) by weight |
|---|---|
| Perilla oil | 20.5 |
| Urtica extract | 13.8 |
| Astragalus extract | 12.2 |
| Nigella oil | 18.5 |
| Soybean oil | 30 |
| Lecithin | 5 |

TABLE 5

| Ingredient | Percentage (%) by weight |
|---|---|
| Perilla extract | 35.3 |
| Urtica extract | 25.4 |
| Astragalus extract | 24.8 |
| Maltodextrine | 14.5 |

TABLE 6

| Ingredient | Percentage (%) by weight |
|---|---|
| Perilla oil | 49.0 |
| Sesame oil | 49.0 |
| Cetirizine hydrochloride | 1.0 |
| Citric acid | 1.0 |

Tables 7 and 8 show an anti-allergic topical gel formulation.

TABLE 7

| Ingredient | Percentage (%) by weight |
|---|---|
| Perilla oil | 15 |
| Sesame oil | 5 |
| Disodium EDTA | 0.1 |
| Menthol | 0.5 |
| Propylene glycol | 12 |
| Carbopol 980 polymer | 0.85 |
| Polysorbate 80 | 1.5 |
| myristate | 2.0 |
| Citric acid | 0.1 |
| Water | 62.95 |

TABLE 8

| Ingredient | Percentage (%) by weight |
|---|---|
| Perilla oil | 15 |
| Sesame oil | 5 |
| Disodium EDTA | 0.1 |
| Menthol | 0.5 |
| Propylene glycol | 12 |
| Carbopol 980 polymer | 0.85 |
| Polysorbate 80 | 1.5 |
| myristate | 2.0 |
| Citric acid | 0.1 |
| Azelastine hydrochloride | 2.0 |
| Water | 60.95 |

Tables 9 and 10 show an anti-allergic lotion formulation.

TABLE 9

| Ingredient | Percentage (%) by weight |
|---|---|
| Perilla oil | 10 |
| Sesame oil | 20 |
| Spearmint oil | 0.1 |
| Water | 62.5 |
| Glycerin | 5 |
| Xanthan gum | 0.5 |
| Carrageenan | 0.3 |
| Tocopherol | 0.5 |
| Phenoxyethanol | 1 |
| Ethylhexylglycerin | 0.1 |

TABLE 10

| Ingredient | Percentage (%) by weight |
|---|---|
| Perilla oil | 5 |
| Sesame oil | 35 |
| Purified water | 40.9 |
| Glycerin | 10 |
| Citric acid | 2 |
| Cetearyl alcohol | 1 |
| Polysorbate 20 | 1 |
| Aloe vera | 5 |
| Vanilla oil | 0.1 |
| Potassium sorbate | 1 |

Table 11 shows an anti-allergic ointment formulation.

TABLE 11

| Ingredient | Percentage (%) by weight |
|---|---|
| Perilla oil | 20 |
| Sesame oil | 20 |
| Menthol | 5 |
| Ointment base | 55 |

Table 12 shows an anti-allergic gel patch formulation.

TABLE 12

| Ingredient | Percentage (%) by weight |
|---|---|
| Perilla oil | 35 |
| Sesame oil | 5 |
| Water | 20 |
| Glycerin | 20 |
| Propylene glycol | 10 |
| Hyaluronic acid | 7 |
| Potassium sorbate | 1 |
| Sodium polyacrylate | 2 |

Tables 13, 14 and 15 show anti-allergic pill formulations in a tablet.

TABLE 13

| Ingredient | Percentage (%) by weight |
|---|---|
| Perilla extract | 38.5 |
| Urtica extract | 30.8 |
| Astragalus extract | 15.4 |
| Croscarmellose sodium | 7.7 |
| Cellulose | 6.8 |
| Silicon dioxide | 0.5 |
| Magnesium stearate | 0.3 |

TABLE 14

| Ingredient | Percentage (%) by weight |
|---|---|
| Perilla extract | 25.3 |
| Urtica extract | 25.3 |
| Astragalus extract | 25.3 |
| Croscarmellose sodium | 1.5 |
| Cellulose microcrystalline | 20.6 |
| Silicon dioxide | 0.5 |
| Magnesium stearate | 1.5 |

TABLE 15

| Ingredient | Percentage (%) by weight |
|---|---|
| Perilla extract | 25.3 |
| Urtica extract | 25.3 |
| Astragalus extract | 25.3 |
| Croscarmellose sodium | 1.5 |
| Cellulose microcrystalline | 20.1 |
| Silicon dioxide | 0.5 |
| Magnesium stearate | 1.5 |
| Bilastine | 0.5 |

1-30. (canceled)

31. A composition comprising a Perilla spp. extract and another plant extract.

32. The composition of claim 31, wherein the other plant extract is an anti-inflammatory and/or an anti-allergic plant extract.

33. The composition of claim 31, wherein the other plant extract is derived from Nigella spp., Urtica spp., Astragalus spp., Petasites spp., Citrus spp., Uncaria spp., Lavandula spp., Mentha spp., Eucalyptus spp., Matricaria spp., Rosmarinus spp., Curcuma spp., Allium spp. or Sesamum spp.

34. The composition of claim 31, wherein the other plant extract is derived from Sesamum spp.

35. The composition of claim 31, wherein the Perilla spp. extract and/or the other plant extract is each, independently, obtained from the seed, root, leaf or whole of the plant.

36. The composition of claim 31, wherein the Perilla spp. extract and/or the other plant extract is each, independently, an oil.

37. The composition of claim 31, wherein the Perilla spp. extract is perilla oil and the other plant extract is sesame oil.

38. The composition of claim 31, wherein the Perilla spp. extract and the other plant extract are present in the composition in a weight ratio ranging from about 1:10 to about 10:1.

39. The composition of claim 31, wherein the composition comprises a tablet, capsule, caplet, drageé, lozenge, granule, powder, pellet, bead, cachet, bolus, elixir, syrup, suspension, spray, emulsion, lotion, cream, solution, tincture ointment, cream, paste, gel, or jelly.

40. The composition of claim 31, wherein the composition inhibits degranulation of the mast cells.

41. The composition of claim 31, wherein the composition inhibits release of histamine from the mast cells.

42. The composition of claim 31, wherein the composition inhibits mast cell activation.

43. The composition of claim 31, wherein the composition prevents and/or treats a disorder associated with mast cell activation and/or mast cell degranulation.

44. The composition of claim 31, wherein the composition prevents and/or treats a mast cell activation disorder, an allergic disorder, allergic asthma, allergic rhinitis, conjunctivitis, angioedema, urticaria, eczema, food allergy, drug allergy, insect allergy, anaphylaxis, an autoimmune disease, a mast cell clonal disorder, mastocytosis, a mast cell neoplastic disorder, mastocytoma, mast cell sarcoma, and/or mast cell leukemia.

45. The composition of claim 31, wherein a concentration of histamine in a tissue of a subject is reduced by at least about 20% after administration of the composition.

46. A method, comprising:
providing a composition comprising a Perilla spp. extract and another plant extract; and
preventing and/or treating a disorder associated with mast cell activation and/or mast cell degranulation by application of the composition.

47. The method of claim 46, further comprising administering the composition to a human or mammal for the preventing or treating.

48. The method of claim 47, wherein the other plant extract is an anti-inflammatory and/or an anti-allergic plant extract.

49. The method of claim 47, wherein the composition inhibits degranulation of the mast cells, and/or inhibits release of histamine from the mast cells.

50. A method, comprising:
providing a composition comprising a Perilla spp. extract and another plant extract; and
reducing a concentration of histamine in a tissue of a subject by at least about 40% by administrating the composition to the tissue.
